(19) United States
(12) Patent Application Publication
(10) Pub. No.: US 2019/0374110 A1
(43) Pub. Date: Dec. 12, 2019

(54) PHOTOACOUSTIC APPARATUS AND CONTROL METHOD

(71) Applicant: CANON KABUSHIKI KAISHA, Tokyo (JP)

(72) Inventor: Nobuhito Suehira, Tokyo (JP)

(21) Appl. No.: 16/427,871

(22) Filed: May 31, 2019

(30) Foreign Application Priority Data
Jun. 8, 2018 (JP) 2018-110402
May 10, 2019 (JP) 2019-089777

(51) Int. Cl. A61B 5/00

(52) U.S. Cl. CPC A61B 5/0095; A61B 2576/00; A61B 5/742; A61B 5/0037

(57) ABSTRACT

A photoacoustic apparatus includes a probe configured to receive an acoustic wave generated from an object in response to irradiation with light and to convert the received acoustic wave into a time-series electric signal; a moving mechanism configured to move the probe with respect to the object; a controller configured to perform the irradiation of the object with light; a determination unit configured to determine whether or not a peak of a waveform of the time-series electric signal is present within a predetermined time range from a timing at which the irradiation with light has been performed, and a processing unit configured to generate a photoacoustic image based on the time-series electric signal.

### BACKGROUND OF THE INVENTION

### Field of the Invention

The present invention relates to an apparatus for acquiring object information.

### Description of the Related Art

Photoacoustic tomography (PAT) is known as a technique for imaging the inside of an object using light.

In photoacoustic tomography, a living organism that is an object is irradiated by pulsed light such as laser light and the light is propagated and diffused inside the living organism. When the light is absorbed by body tissue inside the object, an acoustic wave (typically, an ultrasonic wave) is generated due to thermal expansion. This phenomenon is referred to as a photoacoustic effect and an acoustic wave generated by a photoacoustic effect is referred to as a photoacoustic wave. Since absorption rates of optical energy differ among tissues constituting an object, sound pressure of generated photoacoustic waves also differ. With PAT, by receiving a generated photoacoustic wave with a probe and reconstructing a received signal, characteristic information about the inside of the object can be obtained by an image.

Japanese Patent Application Laid-open No. 2016-053482 discloses a photoacoustic microscope as an apparatus which visualizes object information at high spatial resolution. The photoacoustic microscope is an apparatus capable of acquiring a high-resolution image by focusing light or sound using an optical lens or an acoustic lens.

### SUMMARY OF THE INVENTION

Since a focal spot of a probe of a photoacoustic microscope is generally at a shallow position from a surface of an object, a region of the interest (ROI) of an object is arranged within several hundred micrometers from a receiving surface of a probe. For a purpose of acoustical coupling between the transducer and the object across a predetermined imaging region, a jelly-like acoustic matching agent is applied on a surface of the object. Depending on a thickness of the acoustic matching agent, there may be cases where a position of the region of the interest deviates from a focal spot of the probe. In such a case, object information can no longer be accurately acquired. In a case where it is found that a relative position of the probe with respect to the object was not appropriate based on the photoacoustic image after the completion of generating the photoacoustic image for a several minutes—several ten minutes, the photoacoustic measurement is performed once again, thereby imposing a burden on an examinee. Therefore, desirably, whether or not a relative position of the probe with respect to the object is appropriately set can be determined before starting photoacoustic image generation.

The present invention has been made in consideration of such problems existing in prior art and an object thereof is to readily verify whether or not an object is installed at an appropriate position in a photoacoustic apparatus.

In order to solve the problem described above, a photoacoustic apparatus according to a first aspect of the present invention is the photoacoustic apparatus, comprising: a probe configured to receive an acoustic wave generated from an object in response to irradiation with light and to convert the received acoustic wave into a time-series electric signal; a moving mechanism configured to move the probe with respect to the object; a controller configured to perform the irradiation of the object with light; a determination unit configured to determine whether or not a peak of a waveform of the time-series electric signal is present within a predetermined time range from a timing at which the irradiation with light has been performed, and a processing unit configured to generate a photoacoustic image based on the time-series electric signal.

In addition, a photoacoustic apparatus according to another aspect of the present invention is the photoacoustic apparatus, comprising: a probe configured to receive an acoustic wave generated from an object in response to irradiation with light and to convert the received acoustic wave into a time-series electric signal; and an image generating unit configured to extract a range corresponding to a vicinity of a contact surface with which the probe comes into contact with the object from the time-series electric signal and to generate an image showing a waveform of the electric signal included in the range.

In addition, a control method according to the present invention is the control method performed by a photoacoustic apparatus including a probe which receives an acoustic wave generated from an object in response to irradiation with light and which converts the received acoustic wave into a time-series electric signal and a moving mechanism which moves the probe with respect to the object, the control method comprising: a control step of performing the irradiation of the object with light; a determination step of determining whether or not a peak of a waveform of the time-series electric signal is present within a predetermined time range from a timing at which the irradiation with light has been performed; and a processing step of generating a photoacoustic image based on the time-series electric signal.

In addition, a control method according to another aspect of the present invention is the control method performed by a photoacoustic apparatus including a probe which receives an acoustic wave generated from an object in response to irradiation with light and which converts the received acoustic wave into a time-series electric signal, the control method comprising an image generating step of extracting a range corresponding to a vicinity of a contact surface with which the probe comes into contact with the object from the time-series electric signal and generating an image showing a waveform of the electric signal included in the range.

According to the present invention, whether or not an object is installed at an appropriate position in a photoacoustic apparatus can be verified.

Further features of the present invention will become apparent from the following description of exemplary embodiments with reference to the attached drawings.

### BRIEF DESCRIPTION OF THE DRAWINGS

FIG. 1 is a configuration diagram of a photoacoustic apparatus according to an embodiment;

FIG. 2 is a diagram showing an external appearance of a user interface;

FIGS. 3A to 3C are diagrams showing a waveform of a photoacoustic signal acquired by an acoustic probe;

FIG. 4 is a processing flow chart of the photoacoustic apparatus according to the embodiment;

FIGS. 5A to 5C are diagrams showing a waveform of a photoacoustic signal acquired by an acoustic probe; and

FIGS. 6A and 6B are diagrams showing a waveform of a photoacoustic signal according to a modification.

### DESCRIPTION OF THE EMBODIMENTS

Hereinafter, an embodiment of the present invention will be described with reference to the drawings. However, it is to be understood that dimensions, materials, shapes, relative arrangements, and the like of components described below are intended to be changed as deemed appropriate in accordance with configurations and various conditions of apparatuses to which the present invention is to be applied. Therefore, the scope of the present invention is not intended to be limited to the embodiment described below.

The present invention relates to a technique for detecting an acoustic wave propagating from an object and generating and acquiring characteristic information about the inside of the object. Accordingly, the present invention can be considered a photoacoustic apparatus or a control method thereof. The present invention can also be considered a program that causes an apparatus including hardware resources such as a CPU and a memory to execute these methods or a computer-readable non-transitory storage medium storing the program.

The photoacoustic apparatus according to the embodiment is an apparatus utilizing a photoacoustic effect in which an acoustic wave generated inside an object by irradiating the object with light (an electromagnetic wave) is received and characteristic information about the object is acquired as image data. In this case, characteristic information refers to information on a characteristic value corresponding to each of a plurality of positions inside the object which is generated using a received signal obtained by receiving a photoacoustic wave.

Characteristic information acquired by photoacoustic measurement is a value reflecting an absorption rate of optical energy. For example, characteristic information includes a generation source of acoustic waves generated by light irradiation, initial sound pressure inside an object, a light energy absorption density or an absorption coefficient derived from initial sound pressure, or a concentration of substances constituting tissue.

In addition, information such as a concentration of a substance constituting the object is obtained based on photoacoustic waves generated by light with a plurality of different wavelengths. Such information may be oxygen saturation, a value obtained by weighting oxygen saturation with an intensity of an absorption coefficient or the like, a total hemoglobin concentration, an oxyhemoglobin concentration, or a deoxyhemoglobin concentration. Alternatively, the information may be a glucose concentration, a collagen concentration, a melanin concentration, or a volume fraction of fat or water.

The embodiment described below assumes a photoacoustic imaging apparatus which, by irradiating an object with light having a wavelength that anticipates hemoglobin to be an absorber, acquires data of a distribution and shapes of blood vessels inside the object and data of an oxygen saturation distribution in the blood vessel, and images the data.

A two-dimensional or three-dimensional characteristic information distribution is obtained based on characteristic information at each position in the object. Distribution data may be generated as image data. Characteristic information may be obtained as distribution information of respective positions inside the object instead of as numerical data. In other words, characteristic information may be obtained as distribution information such as an initial sound pressure distribution, an energy absorption density distribution, an absorption coefficient distribution, or an oxygen saturation distribution.

An acoustic wave in the present specification is typically an ultrasonic wave and includes an elastic wave which is also referred to as a sonic wave or a photoacoustic wave. An electric signal transformed from an acoustic wave by a probe or the like is also referred to as an acoustic signal. However, descriptions of an ultrasonic wave or an acoustic wave in the present specification are not intended to limit a wavelength of such elastic waves. An acoustic wave generated by a photoacoustic effect is referred to as a photoacoustic wave or an optical ultrasonic wave. An electric signal derived from a photoacoustic wave is also referred to as a photoacoustic signal. It should be noted that, in the present specification, a photoacoustic signal is a concept encompassing both analog signals and digital signals. Distribution data is also referred to as photoacoustic image data or reconstructed image data.

A photoacoustic apparatus according to the present embodiment is an apparatus which generates information related to optical characteristics of the inside of an object by irradiating the object with pulse light and receiving photoacoustic waves generated inside the object for the purpose of, in particular, observing a relatively shallow range from a body surface.

System Configuration

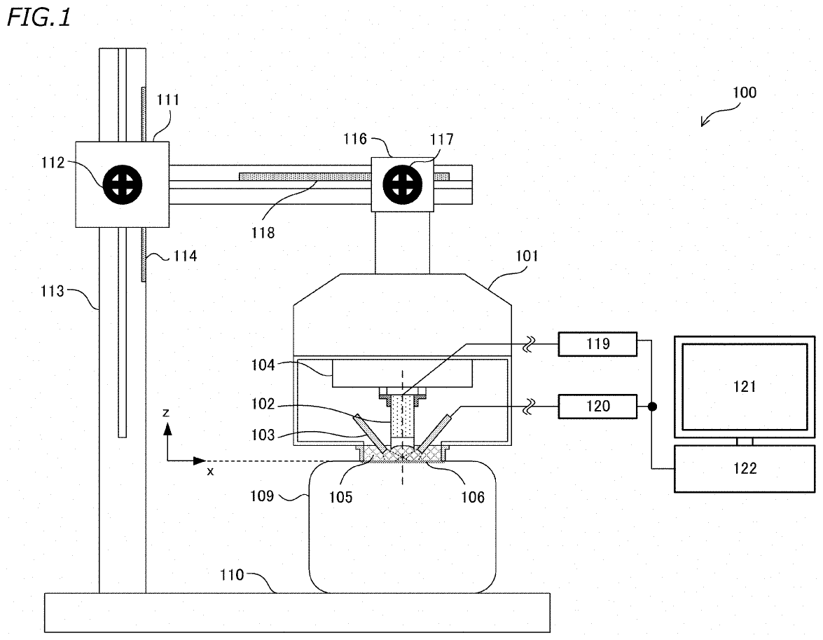

FIG. 1 is a diagram illustrating a configuration of a photoacoustic apparatus 100 according to the present embodiment. The photoacoustic apparatus 100 according to the present embodiment is configured to include a probe unit 101, a probe unit holding mechanism 113, a signal acquiring portion 119, a light source 120, an apparatus control portion 122, and a display apparatus 121.

The probe unit 101 is a unit which irradiates an object with light and which receives an acoustic wave generated from the object. The probe unit 101 is configured to include a light irradiating portion 103 (a controller) for performing irradiation of the object with light, an acoustic probe 102 which performs reception of acoustic waves, and a scanning mechanism 104 (a moving mechanism). The light irradiating portion 103 and the acoustic probe 102 are configured so as to be integrally movable by the scanning mechanism 104. The probe unit 101 and an object 109 come into contact with each other via a biological contact membrane 106. In the following description, the biological contact membrane 106 will be referred to as a “contact surface (of the probe unit with respect to the object)”.

The biological contact membrane 106 is a membrane constituted by polyethylene terephthalate. The biological contact membrane 106 is preferably made of a material having sufficient strength to resist deformation by the object and a characteristic of allowing transmission of light and acoustic waves. In the present embodiment, an opening portion of the biological contact membrane is 30×30 mm². Water 105 that is an acoustic wave propagation medium is stored between the biological contact membrane 106 and the acoustic probe 102. The biological contact membrane 106 preferably has a thickness of around 100 microns in order to avoid multiple reflections of acoustic waves inside the membrane.

The probe unit holding mechanism 113 is a mechanism for holding and moving the probe unit 101. The probe unit holding mechanism 113 is configured to include a Z-axis stage 111 for moving the probe unit 101 in a Z-axis direction and an X-axis stage 116 for moving the probe unit 101 in an X-axis direction.

The Z-axis stage 111 is configured so as to be movable using a Z-axis handle 112. Accordingly, the probe unit 101 can be moved in the Z-axis direction relative to the object 109. A position of the Z-axis stage is detected by a Z-axis encoder 114 and, accordingly, a position of the probe unit in the Z-axis direction can be calculated.

In addition, the X-axis stage 116 is configured so as to be movable using an X-axis handle 117. Accordingly, the probe unit 101 can be moved in the X-axis direction relative to the object 109. A position of the X-axis stage is detected by an X-axis encoder 118 and, accordingly, a position of the probe unit in the X-axis direction can be calculated.

The light source 120 is an apparatus that generates pulse light for irradiating a subject. While the light source 120 is desirably a laser light source in order to obtain a large output, a light-emitting diode or a flash lamp may be used in place of a laser. When using a laser as the light source, various lasers such as a solid-state laser, a gas laser, a dye laser, and a semiconductor laser can be used.

In addition, desirably, a wavelength of the pulse light is a specific wavelength which is absorbed by a specific component among components constituting the object and which enables light to propagate to the inside of the object. Specifically, light with a wavelength of at least 700 nm and not more than 1100 nm is desirably used.

Furthermore, in order to effectively generate a photoacoustic wave, light must be irradiated in a sufficiently short period of time in accordance with thermal characteristics of the object. In a case where the object is a living organism as described in the present embodiment, a pulse width of the pulse light generated from the light source is preferably around 10 to 50 nanoseconds.

Moreover, timings, waveforms, intensity, and the like of light irradiation are controlled by the apparatus control portion 122 to be described later.

In the present embodiment, the pulse width is set to 10 nanoseconds and a repetition frequency is set to 200 Hz. In addition, a YAG laser capable of switching between wavelengths of 532 nm and 1064 nm is used. While 532 nm is a wavelength that is largely absorbed by a living organism, since a target of measurement by the photoacoustic apparatus 100 according to the present embodiment is an approximately 5 mm-range from an object surface, the wavelength can be used. Moreover, blood vessels and melanin can also be identified using the 1064 nm wavelength.

Light emitted from the light source 120 is irradiated to the object 109 using an optical fiber that is the light irradiating portion 103. The optical fiber may be arranged in a ring shape centered on the acoustic probe 102. In addition, spreading light over a relatively wide area is more favorable than focal spotting light with a lens from the perspectives of safety with respect to the subject and expanding a diagnostic area.

The acoustic probe 102 is a unit which receives an acoustic wave arriving from inside an object portion and which converts the acoustic wave into a time-series electric signal. The acoustic probe is also referred to as a probe, an acoustic wave probe, an acoustic wave detecting element, an acoustic wave detector, an acoustic wave receiver, and a transducer.

Since acoustic waves generated by a living organism are ultrasonic waves from 100 kHz to 100 MHz, an element capable of receiving this frequency band is used as the acoustic probe. Specifically, a transducer using a piezoelectric phenomenon, a transducer using optical resonance, a transducer using a variation in capacity, or the like can be used.

In addition, desirably, the acoustic probe to be used has a high receiving sensitivity and a wide frequency band. Specific examples of the acoustic probe include a piezoelectric element using PZT (lead zirconate titanate) or the like, a polymer piezoelectric film material such as PVDF (polyvinylidene fluoride), and acoustic probes using a CMUT (capacitive micromachined ultrasonic transducer) or a Fabry-Perot interferometer. However, the acoustic probe is not limited to these examples and any acoustic probe may be used as long as functions as a probe are satisfied.

The acoustic probe 102 according to the present embodiment is an acoustic focal spotting probe including a PZT element and an acoustic lens. A relative position of a focal spot with respect to the acoustic lens is determined statically for a wavelength of an acoustic wave by shape of a receiving surface, a lens effect of the acoustic lens and an acoustic impedance of elements in a propagation path of the acoustic wave. The elements in a propagation path of the acoustic wave includes an acoustic matching agent and an object. The matching agent generally has an acoustic impedance within a predetermined range so that the matching agent connects (matches) acoustically to the object. Thus, the position of the focal spot is mainly determined by a constitution of the acoustic probe 102. The acoustic probe 102 is capable of efficiently receiving an acoustic wave propagating from a region including the focal spot to the acoustic probe 102 The receiving surface of the acoustic probe 102 according to the present embodiment has a diameter of 6 mm and the transmitting wave of the acoustic probe 102 according to the present embodiment has a diameter of 6 mm and the transmitting wave of the acoustic probe 102 has a central frequency of 50 MHz. The acoustic probe 102 includes an acoustic lens made of quartz glass at a distal end. A numerical aperture of the acoustic lens is 0.6. A resolution in a parallel plane to the receiving surface (an XY plane) depends on a focal diameter on the XY plane. A resolution of the acoustic probe 102 is around 60 μm in the present embodiment. In addition, a resolution in a depth direction (Z direction) is around 80 percent of a detectable wavelength (around 30 μm). A focal spot is positioned 4 mm from the acoustic probe 102 and matches a position of the biological contact membrane 106. There may be cases where the position of the focal spot is favorably arranged closer to a side of the acoustic probe 102 and, in such a case, the position of the focal spot is brought closer by, for example, 0.5 mm.

The signal acquiring portion 119 is a unit which amplifies an analog electric signal acquired by the acoustic probe 102 and which converts the amplified electric signal into a digital signal. The signal acquiring portion 119 may be configured using an amplifier which amplifies a received signal and an A/D converter which digitalizes an analog signal. Alternatively, the signal acquiring portion 119 may be constituted by a plurality of processors or arithmetic circuits.

In the present embodiment, a sampling frequency is set to 500 MHz and the number of samplings is set to 8192. Sampling is started when a predetermined time elapses from generation of a trigger signal that represents a timing of light irradiation. Alternatively, the signal acquiring portion 119 may further include a memory such as FIFO which stores a received signal and an arithmetic circuit such as an FPGA chip. In addition, the apparatus control portion 122 may be realized by a general-purpose computer or an exclusively designed work station.

The apparatus control portion 122 is a unit (the image generating unit according to the present invention) which, by performing a reconstruction process based on the digitalized signal (photoacoustic signal), acquires object information such as a light absorption coefficient and oxygen saturation of the inside of an object. Specifically, a three-dimensional initial sound pressure distribution inside the object is generated from collected electric signals.

In addition, the apparatus control portion 122 generates a three-dimensional light intensity distribution of the inside of the object based on information regarding a light amount irradiated to the object. The three-dimensional light intensity distribution can be acquired by solving a light diffusion equation from information related to a two-dimensional light intensity distribution. In addition, an absorption coefficient distribution inside the object can be obtained using the initial sound pressure distribution inside the object generated from a photoacoustic signal and the three-dimensional light intensity distribution. Furthermore, an oxygen saturation distribution inside the object can be obtained by calculating an absorption coefficient distribution at a plurality of wavelengths.

Moreover, the apparatus control portion 122 may have a function of performing desired processes such as information processing necessary for calculating a light amount distribution and acquiring an optical coefficient of a background, and signal correction.

In addition, the apparatus control portion 122 may acquire, via a display apparatus and an input interface to be described later, instructions related to changing measurement parameters, starting and ending measurements, selecting an image processing method, saving patient information and images, analyzing data, and the like.

Furthermore, the apparatus control portion 122 is also a unit (a determination unit, a processing unit, an adjusting unit) which performs control of various components of the photoacoustic apparatus 100. For example, the apparatus control portion 122 issues commands related to control of the entire apparatus such as control of irradiation of the object with light, reception control of acoustic waves and photoacoustic signals, and movement control of the probe unit.

The apparatus control portion 122 may be constituted by a computer including a CPU, a RAM, a nonvolatile memory, and a control port. Control is performed as a program stored in the nonvolatile memory is executed by the CPU. The apparatus control portion 122 may be realized by a general-purpose computer or an exclusively designed work station. In addition, a unit that serves a calculation function of the apparatus control portion 122 may be constituted by a processor such as a CPU or a GPU and an arithmetic circuit such as an FPGA chip. Such units may not only be constituted by a single processor or a single arithmetic circuit but may also be constituted by a plurality of processors or a plurality of arithmetic circuits.

Furthermore, a unit that serves a storage function of the apparatus control portion 122 may be a non-transitory storage medium such as a ROM, a magnetic disk, or a flash memory or a volatile medium such as a RAM. A storage medium in which the program is to be stored is a non-transitory storage medium. Moreover, the units are not limited to being constituted by a single storage medium and may be constituted by a plurality of storage media. A unit that serves a control function of the apparatus control portion 122 is constituted by an arithmetic element such as a CPU.

The display apparatus 121 is a unit which displays information acquired by the apparatus control portion 122 and processed information based on the acquired information, and is typically a display device. The display apparatus 121 may be a plurality of apparatuses or a single apparatus which is provided with a plurality of display portions and which is capable of parallel display. For the display apparatus 121, an apparatus is desirably used which has a size of 30 inches or larger and a contrast ratio of 1000:1 or higher and which is capable of color display at high resolution.

Next, an example of a user interface for performing measurement by the photoacoustic apparatus 100 according to the present embodiment will be described.

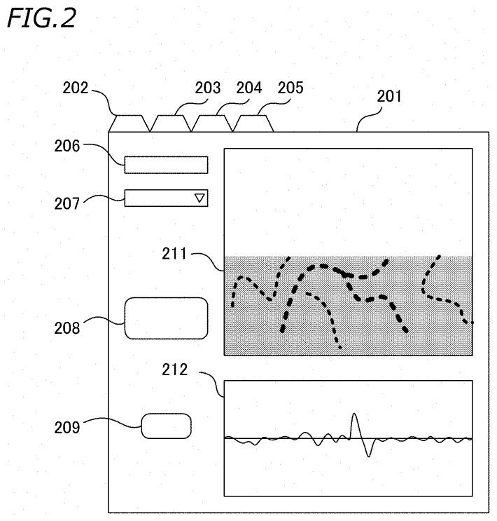

Control by the apparatus control portion 122 is performed by application software. FIG. 2 shows an input interface screen of the application software. The screen is displayed via the display apparatus 121.

A window 201 of the application software has a measurement tab 202, a measurement parameter setting tab 203, a reconstruction tab 204, and a reconstruction parameter setting tab 205, and a user (an operator) of the apparatus selects one of the tabs. Note that FIG. 2 shows a state where the measurement tab 202 has been selected.

When the measurement tab 202 is selected, an input portion 206 for inputting patient information, a measurement mode selection list 207 for selecting a scan range or a measurement time, and an image measurement button 208 for starting a two-dimensional scan become usable.

The measurement mode selection list 207 is a list box for selecting a scan pitch and an imaging size. In the present example, it is assumed that the scan pitch can be selected from 25, 50, and 100 μm, and the imaging size can be selected from 3×3, 5×5, and 10×10 mm². The imaging size refers to a maximum range of an image displayed after reconstruction. Note that the scan ranges are ranges respectively wider by 2 mm than the imaging sizes. This is because generating one pixel worth of data requires peripheral data.

A height evaluation button 209 is a button for evaluating whether or not a height of the probe unit 101 (in other words, a relative position of the biological contact membrane 106 with respect to the object) is appropriate. A specific operation will be described later.

Furthermore, by selecting the measurement tab 202, an image display portion 211 which displays a photoacoustic image and a signal display portion 212 become usable.

When the measurement parameter setting tab 203 is selected, an interface for setting an initial position of the acoustic probe 102, the number of samplings of signals, an irradiation frequency of light, a scan direction, an imaging range of a photoacoustic signal, and the like becomes usable.

In addition, when the reconstruction parameter setting tab 205 is selected, an interface for setting an image resolution to be calculated by reconstruction, an image processing range, a reconstruction algorithm, an image filter, an image output format, and the like becomes usable.

Furthermore, when the reconstruction tab 204 is selected, an interface for generating and displaying an image representing a distribution of light absorption coefficients and an image representing a distribution of oxygen saturation by instructing data selection and reconstruction becomes usable.

Next, a method carried out by the photoacoustic apparatus 100 according to the present embodiment to measure a living organism that is an object will be described.

First, pulsed light emitted from the light source 120 irradiates the object via the light irradiating portion 103. When a part of energy of the light having propagated inside the object is absorbed by a light absorber such as blood, an acoustic wave is generated by the light absorber due to thermal expansion. When cancer exists in the living organism, light is specifically absorbed by new blood vessels in the cancer in a similar manner to blood vessels in other healthy parts and an acoustic wave is generated. A photoacoustic wave generated inside the living organism due to irradiation of light is received by the acoustic probe 102.

In the present embodiment, irradiation of light and acquisition of acoustic waves can be performed while changing a relative positional relationship between the probe unit 101 and the object using the scanning mechanism. In other words, photoacoustic signals can be acquired while irradiating different positions on the object by light a plurality of times.

A signal received by the acoustic probe 102 is converted by the signal acquiring portion 119 and then analyzed by the apparatus control portion 122. An analysis result becomes volume data representing characteristic information (for example, an initial sound pressure distribution or an absorption coefficient distribution) about the inside of the living organism and, after being converted into a two-dimensional image, the converted volume data is output via the display apparatus 121.

The apparatus control portion 122 executes an image reconstruction process using a photoacoustic signal. Known reconstruction methods such as a universal back-projection method and a phasing addition method can be used for image reconstruction. A case where the universal back-projection method is used will now be described. First, an initial sound pressure distribution P(r) generated in photoacoustic measurement is expressed by Expression (1).

$$P ( r ) = {\int }_{{Ω}_{0}} b ( {r}_{0} , t = \vert r - {r}_{0} \vert ) \frac{d {Ω}_{0}}{{Ω}_{0}} \qquad Expression ( 1 )$$

A term b(r₀, t) corresponding to projection data in this case is shown in Expression (2), where p_{d}(r₀, t) denotes a photoacoustic signal detected by the acoustic probe 102, r₀ denotes a position of each acoustic probe 102, t denotes time, and Ω₀ denotes a solid angle of the acoustic probe 102. The initial sound pressure distribution P(r) can be obtained by processing, based on Expression (1), data acquired by the signal acquiring portion 119.

$$b ( {r}_{0} , t ) = 2 {p}_{d} ( {r}_{0} , t ) - 2 t \frac{\partial {p}_{d} ( {r}_{0} , t )}{\partial t} \qquad Expression ( 2 )$$

As described earlier, a photoacoustic signal is a time-series signal and a relative position of each probe with respect to a light absorber can be obtained from an acquisition time and sound velocity. Using information on the relative position of each probe, by projecting the received signal onto an arc centered on a reception position of the probe having output the photoacoustic signal, an image of the absorber is formed.

An absorption coefficient distribution can be calculated from the initial sound pressure distribution P(r). Specifically, sound pressure P(r) generated when the absorber is in response to irradiation with light is expressed by Expression (3).

P(r)=Γ·μₐ(r)·Φ(r) Expression (3)

Γ denotes a Grüneisen coefficient that is an elasticity characteristic value obtained by dividing a product of a coefficient of volumetric expansion (β) and a square of the sound velocity (c) by specific heat (Cp). μₐ denotes an absorption coefficient of the absorber. In addition, Φ(r) denotes a light amount irradiated to the absorber in a localized region. An absorption coefficient distribution μₐ(r) can be obtained by solving Expression (3) for the absorption coefficient. It should be noted that an optical coefficient of the background is sufficiently smaller than the absorption coefficient of the absorber and is not represented in the absorption coefficient distribution.

When attenuation occurs uniformly in a depth direction or the like, the light amount distribution Φ(r) can be represented using a variable z as shown in Expression (4).

Φ=Φ₀ exp(−μ_{eff}z) Expression (4)

Φ₀ denotes a light amount of measurement light on the surface. μ_{eff} denotes an average equivalent attenuation coefficient inside the object. Incidentally, a scattering coefficient and an absorption coefficient of the background in the object can be measured using, for example, a near-infrared spectrometer or the like. In addition, an oxygen saturation distribution inside the object can be obtained by calculating an absorption coefficient distribution at a plurality of wavelengths.

Next, an outline of processes unique to the photoacoustic apparatus 100 according to the present embodiment will be described.

The photoacoustic apparatus 100 according to the present embodiment is configured such that the apparatus control portion 122 is capable of executing a process of generating an image (hereinafter, a signal waveform image) indicating an analog waveform of an acquired photoacoustic signal in addition to a process of generating a reconstructed image based on the acquired photoacoustic signal.

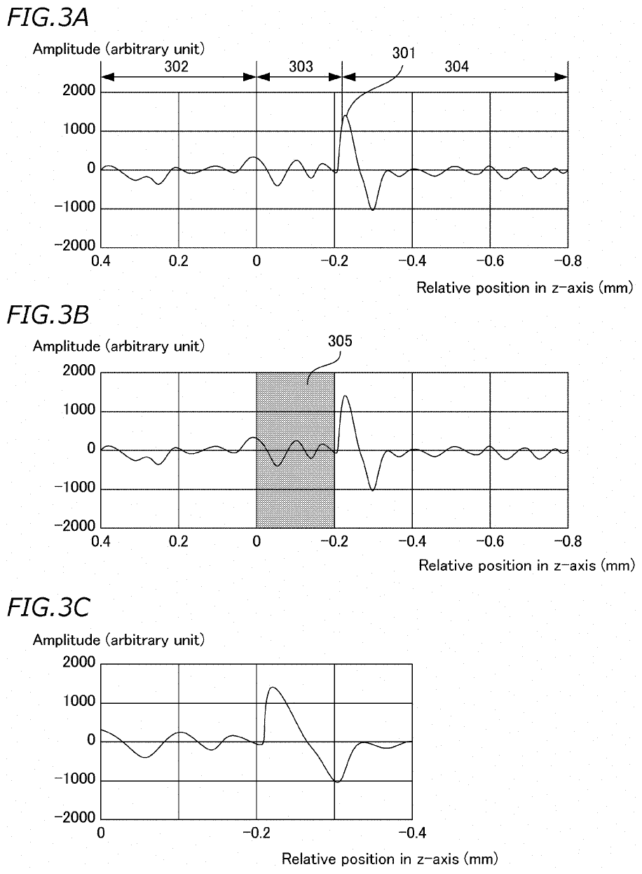

A signal waveform image will now be described. A photoacoustic signal corresponding to an acoustic wave generated inside the object is output as time-series data originating from a timing at which light irradiation is performed. The signal waveform image in the present embodiment is a representation of the time-series data by an image with amplitude as an ordinate and a time axis as an abscissa. FIG. 3A shows an example of the signal waveform image. Propagation time of an acoustic wave is proportional to relative position. Therefore, in the illustrated image, the abscissa is converted to relative position (in millimeter units). The conversion can be performed based on the sound velocity in water which is an acoustic propagation medium. The sound velocity in water can be determined based on a temperature of the water. The abscissas of the respective drawings in FIGS. 3A to 3C can be paraphrased as positions on a propagation path of an acoustic wave from the acoustic probe 102. The abscissas of the respective drawings in FIGS. 3A to 3C can be paraphrased as indicating positions on the propagation path of an acoustic wave from the acoustic probe 102. In addition, the abscissas of the respective drawings in FIGS. 3A to 3C can be paraphrased as indicating positions in a depth direction on the propagation path of an acoustic wave from the acoustic probe 102. Hereinafter, a predetermined range as used in the present embodiment means a predetermined range on a time axis and may be referred to as a predetermined time range.

A position where the biological contact membrane 106 is present is a position at a relative position of 0. In other words, the illustrated signal waveform image can be described as representing an intensity of an acoustic wave generated in a nearby range from +0.4 mm to −0.8 mm based on the biological contact membrane 106.

When sound velocity is assumed to be 1500 m/s and the number of samplings is set to 8192 times, data can be acquired from a region down to a depth of 24.6 mm. However, since a reachable depth of light is around 5 mm and a resolution in the depth direction by one sampling is 3 um, only a range of 400 pixels is considered a display target in the present example.

A reference numeral 303 in FIG. 3A denotes a region from the position of the biological contact membrane 106 to the surface of the object. In addition, a reference numeral 304 denotes a region inside the object. In the present example, an ultrasonic gel with high viscosity is used as an acoustic matching agent with the object. Since there are cases where the ultrasonic gel causes the surface of the object to separate from the biological contact membrane 106 or causes the biological contact membrane 106 to deform, a total width of 1.2 mm is secured in the present example in anticipation of such occurrences. Moreover, a reference numeral 302 denotes a section corresponding to a propagation path of an acoustic wave from a receiving surface of the probe 102 to the biological contact membrane 106. An acoustic lens and an acoustic matching agent (water 105) are positioned as media for propagating an acoustic wave on the propagation path corresponding to the reference numeral 302.

FIG. 3B shows an example in which a region (hereinafter, a target region: in the present example, a region 305 down to −0.2 mm) of a predetermined range from the position of the biological contact membrane 106 is displayed colored and marked. Since photoacoustic signals are intensely generated on a surface of skin, when a peak of a photoacoustic signal deviates from this range, it is presumed that the biological contact membrane and the surface of the object are not in close contact with each other. In this manner, changing a background color of the target region 305 makes it easier to recognize that a signal 301 generated from the skin surface of the object has entered the target region 305.

A position of the surface of the object can be estimated by extracting a range which is constituted by a pair of a signal larger than a positive threshold and a signal smaller than a negative threshold and which is positioned closest to the biological contact membrane 106.

FIG. 3C shows an example displaying only a vicinity of the surface of the biological contact membrane 106. Such a display is favorable when using water as the acoustic matching agent and when the opening portion of the biological contact membrane 106 is relatively small and a position thereof hardly changes. An image corresponding to the whole and an image corresponding to the vicinity of the surface of the biological contact membrane 106 may be respectively displayed independently.

In the present embodiment, an adjustment of the relative position of the probe unit with respect to the object is performed while providing the user with such a signal waveform image. Since a signal waveform image is generated at any time, whether or not the probe unit and the object are in close contact with each other can be determined by continuously observing signal waveform images. In other words, the user of the apparatus can determine a favorable relative position of the probe unit with respect to the object before starting photoacoustic imaging.

In other words, the favorable relative position of the probe with respect to the object is rephrased into at least any one of “home position”, “starting position”, “first relative position”, “preset position” and “predetermined position”.

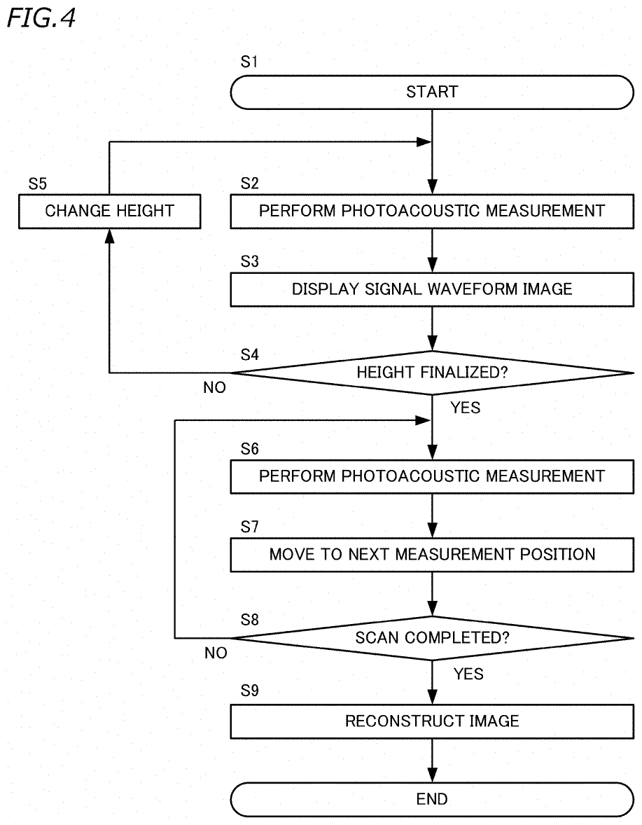

Next, a flow chart of a process performed by the photoacoustic apparatus 100 according to the present embodiment is shown in FIG. 4.

First, in step S1, a measurement is started. In the present step, the user of the apparatus uses the X-axis handle 117 and the Z-axis handle 112 to adjust the probe unit 101 (the biological contact membrane 106) and the object so as to come into close contact with each other. An ultrasonic gel for acoustic matching may be applied between the probe unit 101 and the object 109.

Moreover, the scan pattern, the imaging size (a size on the object to be subjected to imaging), the scan pitch, and the like described earlier may be determined in the present step. In the present embodiment, the imaging size is set to 3×3 mm² and the scan pitch is set to 50 um.

The height evaluation button 209 becomes depressible once preparations are completed, and when the button is depressed, the process makes a transition to step S2.

In step S2, a photoacoustic measurement for evaluating the height of the probe unit 101 (in other words, a relative position of the probe unit 101 with respect to the object) is performed. In the present step, the object 109 is irradiated with pulsed light and a photoacoustic wave having propagated from the object is received. After arriving at the object 109, the irradiated pulsed light is propagated inside the living organism and absorbed by a light absorber. Subsequently, a photoacoustic wave generated from the light absorber is transmitted through the biological contact membrane 106 and received by the acoustic probe 102. The received acoustic wave is converted into an electric signal and transmitted to the apparatus control portion 122 via the signal acquiring portion 119. The obtained photoacoustic signal is associated with an irradiation position of light (a reception position of the acoustic wave) and temporarily stored.

In step S3, a signal waveform image is generated based on the photoacoustic signal obtained in step S2 and the signal waveform image is output via the signal display portion 212.

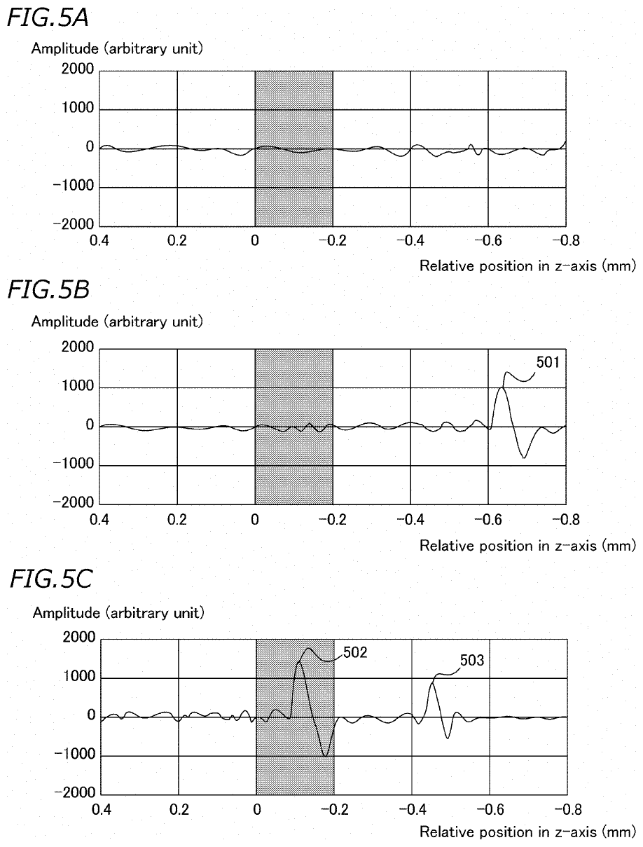

FIGS. 5A to 5C show examples of the photoacoustic signal obtained in step S3. FIG. 5A represents an example of a case where the surface of the object 109 is not present within a range from +0.4 mm to −0.8 mm based on the biological contact membrane 106. When such a waveform is obtained, the probe unit 101 must be moved closer to the object.

FIG. 5B represents an example of a case where a peak (reference numeral 501) of a signal generated from the surface of the object 109 is present within the range from +0.4 mm to −0.8 mm. When such a waveform is obtained, since the object surface is not within the target region, the probe unit 101 must be moved further closer to the object.

FIG. 5C represents an example of a case where a peak (reference numeral 502) of a signal generated from the surface of the object 109 is present within the target region. Reference numeral 503 denotes a photoacoustic signal generated from a blood vessel or the like inside the object. Amplitudes of the peaks denoted by the reference numerals 502 and 503 increase closer to the biological contact membrane 106 which is near the focal spot.

It should be noted that the acquired photoacoustic signal includes a signal generated due to external light, randomly-generated electrical noise, and the like in addition to a signal originating from the living organism. However, with a signal originating from the living organism, since a peak position of the signal changes as the height of the probe unit 101 changes, the signal can be readily determined.

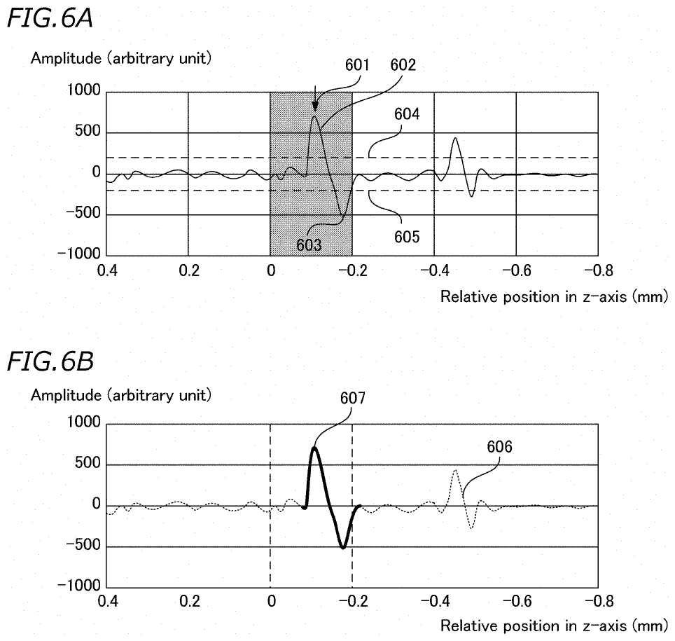

When generating a signal waveform image, a range corresponding to the surface of the object or a position corresponding to the surface of the object may be displayed highlighted. FIG. 6A shows an example of a case where a position of the surface of the object is displayed superimposed by a marker 601. In the present example, a color of the background is changed with respect to a range corresponding to the target region.

A position of the surface of the object can be estimated by extracting a range which includes a pair of a signal 602 larger than a positive threshold 604 and a signal 603 smaller than a negative threshold 605 and which is positioned closest to the biological contact membrane 106.

Since a signal originating from the object moves so as to follow a change to the height of the probe unit, the signal can be distinguished from noise.

FIG. 6B shows an example of a signal waveform image generated with a different design. In the present example, the target region is enclosed by a dash line. In addition, in a waveform of a photoacoustic signal, a portion corresponding to the surface of the object is displayed by a solid line 607 and other portions are displayed by a dotted line.

In step S4, the user of the apparatus determines whether or not the probe unit is at a desired height while checking a signal waveform image. A state where the probe unit 101 is at a desired height is a state where the surface of the object is present within the target region. In this state, it can be described that the biological contact membrane 106 and the surface of the object are in proper contact with each other.

In a case where the probe unit is not at its desired height, in step S5, the user changes the height of the probe unit 101. The user changes the height by operating the Z-axis handle 112. Alternatively, the height may be changed using a motor-driven stage. Control of the motor-driven stage can be performed using a joystick controller, a push button switch, a keyboard, a foot pedal, or the like.

After changing the height, when the user depresses the height evaluation button 209, the process once again makes a transition to step S2.

In step S4, when it is determined that the probe unit 101 is at its desired height, inputting the determination (for example, the user depressing the image measurement button 208) causes the height of the probe unit to be finalized and the process advances to step S6. Moreover, when the apparatus determines that the surface of the object is in proper contact with the target region, the determination may be displayed by a popup window or the like. On the other hand, when the apparatus determines that the surface of the object is not in proper contact with the target region, a warning may be displayed or the measurement may be aborted.

In step S6, a photoacoustic measurement is started. First, a command to drive the scanning mechanism 104 is transmitted from the apparatus control portion 122 and the stage is moved to a scan start position. Subsequently, the object 109 is irradiated with pulsed light and a photoacoustic wave from the living organism is acquired. Light irradiation is performed at a repetition frequency of 200 Hz.

In step S7, using the scanning mechanism 104, the acoustic probe 102 and the light irradiating portion 103 are moved in a scanning direction by one step. The photoacoustic apparatus 100 according to the present embodiment is capable of irradiating light and receiving acoustic waves at a plurality of locations while moving the acoustic probe 102 and the light irradiating portion 103. The scanning direction is determined based on a scan pattern determined in advance. For example, when performing a scan by combining a movement in a main scanning direction (X axis) and a movement in a sub-scanning direction (Y axis), in step S7, either the movement in the main scanning direction or the movement in the sub-scanning direction is performed. As the scan progresses, an object image of a scan region is displayed on the image display portion 211 as, for example, an MIP (Maximum Intensity Projection) image. Subsequently, after measurements of all scan regions are completed, the MIP image may be switched to a three-dimensional reconstructed image.

In step S8, a determination is made as to whether scans with respect to an area that is an imaging target have been completed. For example, when an entire scan range is 7×7 mm², a stroke in a main scanning direction is 7 mm, and the pitch is set to 50 um, the number of scans in one measurement in the main scanning direction is 141 times. In addition, when a stroke in a sub-scanning direction is 7 mm, a scan is completed by repetitively performing 141 main scans.

When the scan is not completed, the process returns to step S6. When the scan is completed, laser irradiation is ended and the stage is returned to the initial position.

In step S9, reconstruction of an image is performed. The reconstructed image is output to the display apparatus 121. The generated image is an image representing initial sound pressure, an image representing an absorption coefficient, an image representing oxygen saturation, or the like. Alternatively, a maximum intensity projection (MIP) image may be generated and displayed from a plurality of tomographic images. Accordingly, how blood vessels connect to each other can be more easily comprehended.

As described above, the photoacoustic apparatus 100 according to the present embodiment evaluates a relative position of the probe unit with respect to the object before starting a photoacoustic measurement of the object by generating an image showing a waveform of photoacoustic signals obtained in a time series and outputting the generated image. According to the aspect described above, whether or not the relative position of the probe unit with respect to the object is appropriate can be determined based on a position where a peak of the waveform appears. In other words, whether or not a photoacoustic measurement with respect to the object can be correctly performed can be determined in advance.

The evaluation of the relative position of the probe unit can be performed by one light irradiation. In other words, since there is no longer a need to perform light irradiation a plurality of times or to wait until an image is reconstructed, even in the case of a single transducer, whether or not a state of arrangement of the object is correct can be determined in a short period of time while adjusting a height of the transducer.

First Modification

While the user of the apparatus determines whether or not the height of the probe unit 101 is appropriate in step S4 in the embodiment described above, whether or not the surface of the object is in proper contact with the target region may be determined by the apparatus by detecting a peak position of a signal waveform.

For example, a pair of signals which include a signal larger than a positive threshold and a signal smaller than a negative threshold and which are positioned closest to the biological contact membrane 106 is extracted from photoacoustic signals obtained in a time series, and a position of the surface of the object is estimated based on a result of the extraction. Subsequently, based on whether or not the estimated surface position is present within a predetermined range from a position based on a contact surface of the probe (the biological contact membrane), whether or not the height of the probe unit (in other words, the relative position of the probe unit with respect to the object) is appropriate can be determined. Moreover, a result of the determination may be notified to the user of the apparatus. In addition, in doing so, a direction and a relative position in which the probe unit should be moved may be notified to the user. Furthermore, when the height of the probe unit is appropriate, the height may be stored to be used in subsequent measurements.

Second Modification

While the user of the apparatus changes the height of the probe unit 101 in step S5 in the embodiment described above, when the Z-axis stage 111 is a motor-driven stage, height adjustment may be performed automatically. For example, in step S5, a direction in which the probe unit 101 is to be moved may be determined and the probe unit 101 may be driven only by a predetermined relative position. Alternatively, the depression of the height evaluation button 209 may also be automated, and the process of steps S2 to S5 may be automatically repeated until the probe unit reaches an appropriate height.

According to the aspect described above, the relative position of the probe unit with respect to the object can be automatically adjusted to an appropriate relative position based on a plurality of photoacoustic signals obtained by light irradiation of a plurality of times.

Other Embodiments

It is to be understood that the description of the embodiment merely presents an example of the present invention and, as such, the present invention can be implemented by appropriately modifying or combining the embodiment without departing from the spirit and the scope of the invention.

For example, the present invention may be implemented as a photoacoustic apparatus 100 which includes at least a part of the units described above. In addition, the present invention may also be implemented as a control method which includes at least a part of the processes described above. The processes described above may be implemented in any combination thereof insofar as technical contradictions do not arise.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2018-110402, filed on Jun. 8, 2018 and Japanese Patent Application No. 2019-089777, filed on May 10, 2019, which are hereby incorporated by reference herein in their entirety.

What is claimed is:

1. A photoacoustic apparatus, comprising:
a probe configured to receive an acoustic wave generated from an object in response to irradiation with light and to convert the received acoustic wave into a time-series electric signal;
a moving mechanism configured to move the probe with respect to the object;
a controller configured to perform the irradiation of the object with light;
a determination unit configured to determine whether or not a peak of a waveform of the time-series electric signal is present within a predetermined time range from a timing at which the irradiation with light has been performed, and
a processing unit configured to generate a photoacoustic image based on the time-series electric signal.

2. The photoacoustic apparatus according to claim 1, wherein
the controller performs the irradiation of the object with light a plurality of times, and
the photoacoustic apparatus further comprises a decision unit configured to determine, based on waveforms of a plurality of time-series electric signals obtained while changing a relative position of the probe with respect to the object, a first relative position that is a relative position to be set before the processing unit starts the photoacoustic image generation of the object.

3. The photoacoustic apparatus according to claim 2, wherein
the decision unit adopts, in a case where a peak of a waveform of the time-series electric signal is present within a predetermined time range from a timing at which the irradiation with light has been performed, a relative position of the probe with respect to the object upon performing the irradiation with light as the first relative position.

4. The photoacoustic apparatus according to claim 1, wherein
the probe has a contact surface which comes into contact with the object, and
the predetermined time range is set based on a position of the contact surface.

5. The photoacoustic apparatus according to claim 1, further comprising
an adjusting unit configured to, in a case where the peak of the waveform of the time-series electric signal is not present within the predetermined time range, notify a user that a relative position of the probe with respect to the object needs to be changed.

6. The photoacoustic apparatus according to claim 1, further comprising
an adjusting unit configured to, in a case where a peak of the waveform of the time-series electric signal is not present within the predetermined time range, change a relative position of the probe with respect to the object by using the moving mechanism.

7. The photoacoustic apparatus according to claim 6, wherein
the adjusting unit changes the relative position to a shorter relative position in a case where a peak of the waveform is present after the predetermined time range, and changes the relative position to a longer relative position in a case where a peak of the waveform is present before the predetermined time range.

8. A photoacoustic apparatus, comprising:
a probe configured to receive an acoustic wave generated from an object in response to irradiation with light and to convert the received acoustic wave into a time-series electric signal; and
an image generating unit configured to extract a range corresponding to a vicinity of a contact surface with which the probe comes into contact with the object from the time-series electric signal and to generate an image showing a waveform of the electric signal included in the range.

9. The photoacoustic apparatus according to claim 8, wherein
the image generating unit superimposes a marker corresponding to a position of the contact surface onto the image.

10. The photoacoustic apparatus according to claim 8, wherein
the image generating unit estimates a surface position of the object based on a waveform of the electric signal, and superimposes a marker corresponding to the surface position onto the image.

11. The photoacoustic apparatus according to claim 1, wherein
the probe is an acoustic focal spotting probe.

12. The photoacoustic apparatus according to claim 8, wherein
the probe is an acoustic focal spotting probe.

13. A control method performed by a photoacoustic apparatus including a probe which receives an acoustic wave generated from an object in response to irradiation with light and which converts the received acoustic wave into a time-series electric signal and a moving mechanism which moves the probe with respect to the object, the control method comprising:
a control step of performing the irradiation of the object with light;
a determination step of determining whether or not a peak of a waveform of the time-series electric signal is present within a predetermined time range from a timing at which the irradiation with light has been performed; and
a processing step of generating a photoacoustic image based on the time-series electric signal.

14. The control method according to claim 13, wherein
in the control step, the irradiation of the object with light is performed a plurality of times, and
the control method further comprises a decision step of determining, based on waveforms of a plurality of time-series electric signals obtained while changing a relative position of the probe with respect to the object, a first relative position that is a relative position to be set before the processing step.

15. A control method performed by a photoacoustic apparatus including a probe which receives an acoustic wave generated from an object in response to irradiation with light and which converts the received acoustic wave into a time-series electric signal, the control method comprising
an image generating step of extracting a range corresponding to a vicinity of a contact surface with which the probe comes into contact with the object from the time-series electric signal and generating an image showing a waveform of the electric signal included in the range.
